(19) United States
(12) Patent Application Publication
(10) Pub. No.: US 2026/0301923 A1
(43) Pub. Date: Oct. 1, 2026

(54) ONLINE REPORT GENERATION METHOD AND ELECTRONIC DEVICE

(71) Applicants: Acer Incorporated, New Taipei City (TW); Acer Medical Inc., NEW TAIPEI CITY (TW)

(72) Inventors: An-Chih Huang, New Taipei City (TW); Meng-Che Cheng, NEW TAIPEI CITY (TW)

(73) Assignees: Acer Incorporated, New Taipei City (TW); Acer Medical Inc., NEW TAIPEI CITY (TW)

(21) Appl. No.: 19/224,916

(22) Filed: Jun. 2, 2025

(30) Foreign Application Priority Data
Mar. 28, 2025 (TW) 114111980

(51) Int. Cl. G16H 30/40; G06T 7/00; G16H 10/60; G16H 30/20

(52) U.S. Cl. CPC G16H 30/40; G06T 7/0012; G16H 10/60; G16H 30/20

(57) ABSTRACT

An online report generation method and an electronic device are disclosed. The method includes: detecting an upload request from a terminal device, wherein the upload request is configured to upload image data and label data; obtaining the image data and label data according to the upload request; obtaining user identity identification information corresponding to the image data according to the label data; confirming whether the image data meets an automatic detection condition according to the user identity identification information; in response to the image data meeting the automatic detection condition, enabling an automatic detection model to perform automatic detection on the image data; and generating an online detection report corresponding to the image data according to a detection result.

### CROSS-REFERENCE TO RELATED APPLICATION

This application claims the priority benefit of Taiwan application serial no. 114111980, filed on March 28, 2025. The entirety of the above-mentioned patent application is hereby incorporated by reference herein and made a part of this specification.

### BACKGROUND

### TECHNICAL FIELD

The disclosure relates to an online report generation method and an electronic device.

### DESCRIPTION OF RELATED ART

With the continuous advancement of technology, the maintenance of human health has gained increasing importance. Generally, several basic elements used to evaluate human health include blood pressure, blood sugar, blood lipids, and bone density, etc. Blood pressure, blood sugar, and blood lipids may be obtained through simple examinations of the subject (e.g., blood pressure measurement or blood analysis), whereas bone density testing is more complex, requiring, for example, X-ray imaging of the bones of the subject, followed by professional assessment of the X-ray images by a physician to confirm the bone density score of the subject.

However, although certain types of image detection models may be used to assist in automatic image analysis for detecting target objects within images, in practice, there remains a lack of a comprehensive and efficient online report generation solution to assist professional or non-professional individuals in evaluating the physiological structure status of users.

An online report generation method and an electronic device, which may improve the above-mentioned problem, are provided in the disclosure.

An online report generation method is provided in the disclosure, the online report generation method comprises the following operation. An upload request is detected from a terminal device, in which the upload request is configured to upload image data and label data. The image data and the label data are obtained according to the upload request. User identity identification information corresponding to the image data is obtained according to the label data. Whether the image data meets an automatic detection condition is confirmed according to the user identity identification information. In response to the image data meeting the automatic detection condition, an automatic detection model is enabled to perform automatic detection on the image data. An online detection report corresponding to the image data is generated according to a detection result.

An electronic device including a storage device and a processor is provided in the disclosure. The storage device is configured to store an automatic detection model. The processor is coupled to the storage device and is configured to execute the following operation. An upload request is detected from a terminal device, in which the upload request is configured to upload image data and label data. The image data and the label data are obtained according to the upload request. User identity identification information corresponding to the image data is obtained according to the label data. Whether the image data meets an automatic detection condition is confirmed according to the user identity identification information. In response to the image data meeting the automatic detection condition, the automatic detection model is enabled to perform automatic detection on the image data. An online detection report corresponding to the image data is generated according to a detection result.

Based on the above, after detecting an upload request from a terminal device, the image data and the label data may be obtained according to the upload request. User identity identification information corresponding to the image data may be obtained according to the label data, and the user identity identification information may be configured to confirm whether the image data meets the automatic detection condition. In response to the image data meeting the automatic detection condition, the automatic detection model may be enabled to perform automatic detection on the image data. An online detection report corresponding to the image data may be automatically generated according to the detection result. Thus, the disclosure provides a comprehensive and efficient online report generation solution to assist professionals or non-professionals in evaluating the physiological structure status of the user.

### BRIEF DESCRIPTION OF THE DRAWINGS

FIG. 1 is a schematic diagram of an electronic device according to an embodiment of the disclosure.

FIG. 2 to FIG. 7 are flowcharts of an online report generation method according to an embodiment of the disclosure.

### DETAILED DESCRIPTION OF DISCLOSED EMBODIMENTS

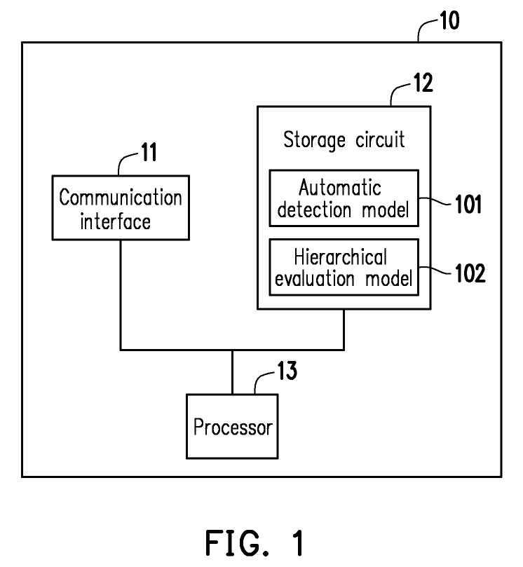

FIG. 1 is a schematic diagram of an electronic device according to an embodiment of the disclosure. Referring to FIG. 1, the electronic device 10 may be various types of electronic devices with communication and data processing capabilities, such as a smartphone, a tablet, a laptop, a desktop computer, an industrial computer, an in-vehicle computer, a server, or a gaming console, and the type of the electronic device 10 is not limited thereto.

The electronic device 10 includes a communication interface 11, a storage device 12 and a processor 13. The communication interface 11 is configured to execute wired or wireless communication functions to transmit signals via wired or wireless means. For example, the communication interface 11 may include a communication circuit and/or a communication interface card (e.g., a network interface card). The communication interface 11 may support wireless communication standards such as WiFi, Bluetooth, near-field communication (NFC), 3G, 4G or 5G, or wired communication standards such as Ethernet, to transmit signals. Alternatively, the communication interface 11 may also support other communication standards, which is not limited in the disclosure. However, the disclosure does not limit the number and type of the communication interface 11.

The storage device 12 is configured to store data. For example, the storage device 12 may include a volatile storage circuit and a non-volatile storage circuit. The volatile storage circuit is configured to volatilely store data. For example, the volatile storage circuit may include random access memory (RAM) or similar volatile storage media. The non-volatile storage circuit is configured to non-volatilely store data. For example, the non-volatile storage circuit may include read only memory (ROM), solid state disk (SSD), conventional hard disk drive (HDD), or similar non-volatile storage media. However, the disclosure does not limit the number and type of the storage device 12.

The processor 13 is coupled to the communication interface 11 and the storage device 12. The processor 13 is responsible for the whole or a part of the operation of the electronic device 10. For example, the processor 13 may include a central processing unit (CPU), a graphics processing unit (GPU), or other programmable general-purpose or special-purpose microprocessor, a digital signal processor (DSP), a programmable controller, an application specific integrated circuit (ASIC), a programmable logic device (PLD), or other similar devices, or a combination of these devices.

In one embodiment, the processor 13 may also include specialized processors to assist in the execution of logical operations (e.g., neural network computations and/or image processing), such as a vision processing unit (VPU), a neural network processing unit (NPU) and/or a tensor processing unit (TPU). However, the disclosure does not limit the number and type of the processor 13.

In one embodiment, the electronic device 10 may further include various input/output devices or peripheral devices such as a power management circuit, a mouse, a keyboard, a display, a speaker and/or a microphone, and the types of the input/output interfaces and peripheral devices are not limited thereto.

In one embodiment, the storage device 12 stores an automatic detection model 101. The automatic detection model 101 may be configured to execute logical operations (e.g., neural network operations and/or image processing). For example, the automatic detection model 101 may adopt a convolutional neural network (CNN), a recurrent neural network (RNN), a long short term memory model (LSTM), a deep neural network (DNN) or other types of logical operation architectures to implement the required functions. However, the disclosure does not limit the number and type of the automatic detection model 101.

In one embodiment, the processor 13 may detect an upload request from the terminal device through the communication interface 11. For example, the terminal device may transmit the upload request to the electronic device 10 via wired or wireless communication. The communication interface 11 may receive the upload request via wired or wireless communication.

In one embodiment, the upload request is configured to upload image data and label data. For example, the image data may include image data of X-ray images. For example, the image data may be obtained by taking images of a specific body part of a user (also referred to as a target user) using an X-ray image capturing device (e.g., an X-ray machine). For example, the specific body part may include the hands, back, waist, chest, abdomen, hips or legs of the target user, and the specific body part is not limited to the above. In addition, the label data carries identity identification information (also referred to as user identity identification information) corresponding to the image data.

In one embodiment, the label data is embedded within the image data. In one embodiment, the label data is independent of the image data.

In one embodiment, the image data and the label data comply with the digital imaging and communications in medicine (DICOM) protocol. The DICOM protocol is a universal standard protocol that may be used for processing, storing, printing and transmitting images (especially medical images). The DICOM protocol includes file format definitions and network communication protocols. For example, the DICOM protocol is an application protocol based on TCP/IP, which uses TCP/IP to connect various systems. However, in one embodiment, the image data and the label data may also comply with other types of communication protocols, and the disclosure is not limited thereto.

In one embodiment, after detecting an upload request from a terminal device, the processor 13 may obtain the image data and the label data according to the upload request. For example, after detecting the upload request from the terminal device, the processor 13 may continue to receive the image data and the label data from the terminal device through the communication interface 11.

In one embodiment, after starting to receive the image data (and the label data), the processor 13 may determine whether an upload overdue event corresponding to the image data occurs. If an upload overdue event corresponding to the image data occurs, the processor 13 may automatically generate a prompt message (also referred to as a first prompt message) and suspend the processing of the image data. Then, the processor 13 may transmit the first prompt message to the terminal device through the communication interface 11. After the terminal device receives the first prompt message, the terminal device may present the first prompt message through a display of the terminal device to remind the user of the terminal device that an upload overdue event corresponding to the image data has occurred. However, if the upload overdue event corresponding to the image data does not occur, the processor 13 may not generate the first prompt message.

In one embodiment, after obtaining the image data (and the label data), the processor 13 may further determine whether the file format of the image data complies with the specifications. For example, the processor 13 may determine whether the image data is a DICOM file. If the image data is a DICOM file, the processor 13 may determine that the file format of the image data complies with the specifications. However, if the image data is not a DICOM file, the processor 13 may determine that the file format of the image data does not comply with the specifications. In one embodiment, the processor 13 may determine whether the image data is a file of another type to confirm whether the file format of the image data complies with the specifications.

In one embodiment, if it is determined that the file format of the image data does not comply with the specifications, the processor 13 may automatically generate a prompt message (also referred to as a second prompt message) and suspend the processing of the image data. Then, the processor 13 may transmit the second prompt message to the terminal device through the communication interface 11. After the terminal device receives the second prompt message, the terminal device may present the second prompt message through the display of the terminal device to remind the user of the terminal device that the file format of the currently uploaded image data does not comply with the specifications. However, if the file format of the image data complies with the specifications, the processor 13 may not generate the second prompt message.

In one embodiment, after obtaining the image data (and the label data), the processor 13 may further determine whether the image resolution of the image data is sufficient. For example, the processor 13 may determine whether the image resolution of the image data is higher than or not lower than a preset resolution. If the image resolution of the image data is higher than or not lower than the preset resolution, the processor 13 may determine that the image resolution of the image data is sufficient. However, if the image resolution of the image data is not higher than or lower than the preset resolution, the processor 13 may determine that the image resolution of the image data is insufficient. For example, the preset resolution may be set according to practical requirements, and the disclosure is not limited thereto.

In one embodiment, if it is determined that the image resolution of the image data is insufficient, the processor 13 may automatically generate a prompt message (also referred to as a third prompt message) and suspend the processing of the image data. Then, the processor 13 may transmit the third prompt message to the terminal device through the communication interface 11. After the terminal device receives the third prompt message, the terminal device may present the third prompt message through the display of the terminal device to remind the user of the terminal device that the image resolution of the currently uploaded image data is insufficient. However, if the image resolution of the image data is sufficient, the processor 13 may not generate the third prompt message.

In one embodiment, after obtaining the image data (and the label data), the processor 13 may further determine whether a key object is in the image data. The number of the key object may be one or more. For example, the key object may be the bones or other organs at a specific part in the human body. For example, the bone at the specific part may be the last thoracic vertebra (usually marked as T12) and the first lumbar vertebra (usually marked as L1) of the human body, but the disclosure is not limited thereto. In one embodiment, the processor 13 may analyze the image data through a trained image recognition model to detect whether a key object is in the image data. For example, the image recognition model may be stored in the storage device 12. How to train an image recognition model to detect key objects in the image data is a prior art and is not elaborated herein.

In one embodiment, if the key object is not in the image data, the processor 13 automatically generates a prompt message (also referred to as a fourth prompt message) and suspends the processing of the image data. Then, the processor 13 may transmit the fourth prompt message to the terminal device through the communication interface 11. After the terminal device receives the fourth prompt message, the terminal device may present the fourth prompt message through the display of the terminal device to remind the user of the terminal device that the required key object is not in the currently uploaded image data. However, if the key object is in the image data, the processor 13 may not generate the fourth prompt message.

In one embodiment, after obtaining the label data, the processor 13 may obtain user identity identification information corresponding to the image data according to the label data. For example, the processor 13 may extract the user identity identification information from the label data.

In one embodiment, the user identity identification information includes physiological description data related to the target user. For example, the physiological description data may be configured to describe the medical record number, the name, the birthday, the gender, and the image capture date of the image data of the target user. In addition, the physiological description data may also include other useful data related to the target user, which is not limited by the disclosure. In one embodiment, the processor 13 may extract the physiological description data from the user identity identification information.

In one embodiment, the processor 13 may confirm a data parsing format corresponding to the label data according to the upload request. For example, the data parsing format may correspond to the DICOM protocol or other communication protocols. Then, the processor 13 may extract the user identity identification information from the label data based on the data parsing format. For example, the processor 13 may sequentially extract physiological description data describing the medical record number, the name, the birthday, the gender, and the image capture date of the image data of the target user from the label data based on the data parsing format.

In one embodiment, after obtaining the user identity identification information, the processor 13 may further transmit the user identity identification information (e.g., the physiological description data) to the terminal device via the communication interface 11. The terminal device may present the user identity identification information through the display of the terminal device. For example, the user identity identification information may be presented in an operation interface of the display of the terminal device.

In one embodiment, the user of the terminal device may modify or update the user identity identification information through the terminal device (e.g., the operation interface). In one embodiment, the modified or updated user identity identification information may be sent back to the electronic device 10 to replace the original user identity identification information.

In one embodiment, after obtaining the user identity identification information, the processor 13 may confirm whether the image data meets the detection condition (also referred to as the automatic detection condition) according to the user identity identification information. In response to the image data meeting the automatic detection condition, the processor 13 may enable (e.g., trigger) the automatic detection model 101 to perform automatic detection on the image data. Then, the processor 13 may generate an online detection report corresponding to the image data according to the detection result of the automatic detection. However, if the image data does not meet the automatic detection condition, the processor 13 may not enable (e.g., not trigger) the automatic detection model 101. If the automatic detection model 101 is not enabled (e.g., triggered), the automatic detection model 101 does not perform the automatic detection on the image data.

In one embodiment, the processor 13 may confirm whether the physiological description data meets a classification condition (also referred to as a first classification condition) according to the user identity identification information. If the physiological description data meets the first classification condition, the processor 13 may determine that the image data meets the automatic detection condition. However, if the physiological description data does not meet the first classification condition, the processor 13 may determine that the image data does not meet the automatic detection condition.

In one embodiment, the processor 13 may estimate the age of the target user according to the user identity identification information (or the physiological description data). For example, the processor 13 may subtract the birthday of the target user described in the physiological description data from the image capture date of the image data described in the physiological description data to obtain the age of the target user. For example, assuming that the image capture date of image data described in the physiological description data is March 10, 2025, and the birthday of the target user described in the physiological description data is January 1, 2001, the processor 13 may determine the age of the target user (e.g., 24 years old) according to the difference in years between the image capture date and the birthday of the target user (e.g., 24 years).

In one embodiment, after estimating the age of the target user, the processor 13 may determine whether the age of the target user is greater than or not less than a preset age (also referred to as a first preset age). If the age of the target user is greater than or not less than the first preset age, the processor 13 may determine that the physiological description data meets the first classification condition. However, if the age of the target user is not greater than or less than the first preset age, the processor 13 may determine that the physiological description data does not meet the first classification condition. For example, the first preset age may be 20 years old. It should be noted that the first preset age may be adjusted according to practical requirements, and the disclosure is not limited thereto.

In one embodiment, after confirming that the image data meets the automatic detection condition and enabling the automatic detection model 101, the automatic detection model 101 may generate parameter data based on the image data. For example, after enabling the automatic detection model 101, the automatic detection model 101 may automatically analyze the pixel values of multiple pixel positions in the image data and generate the parameter data according to the analysis result. For example, the parameter data may include one or more parameter values.

In one embodiment, the parameter data may reflect the physiological structure status of the target user. In one embodiment, the physiological structure status may include bone density. For example, the parameter value in the parameter data may reflect the bone density of the target user. For example, the physical meaning of bone density is how many grams of bone mineral are contained in every square centimeter of bone. Alternatively, in one embodiment, the physiological structure status may also include bone spacing, etc., which is not limited by the disclosure. In one embodiment, the processor 13 may generate a detection result of the automatic detection according to the parameter data.

In one embodiment, after obtaining the parameter data, the processor 13 may also input the parameter data into the hierarchical evaluation model 102 according to the user identity identification information to obtain the detection result of the automatic detection. For example, the hierarchical evaluation model 102 is stored in the storage device 12. In particular, the detection result may reflect an evaluation result of the physiological structure status based on at least one of multiple evaluation rules.

In one embodiment, the processor 13 may confirm whether the physiological description data meets another classification condition (also referred to as the second classification condition) according to the user identity identification information. If the physiological description data meets the second classification condition, the hierarchical evaluation model 102 may evaluate the physiological structure status of the target user based on the parameter data and a specific evaluation rule (also referred to as the first evaluation rule) among the multiple evaluation rules to obtain a detection result (also referred to as the first detection result). However, if the physiological description data does not meet the second classification condition, the hierarchical evaluation model 102 may evaluate the physiological structure status of the target user based on the parameter data and another evaluation rule (also referred to as the second evaluation rule) among the multiple evaluation rules to obtain a detection result (also referred to as the second detection result). The second evaluation rule is different from the first evaluation rule.

In one embodiment, the processor 13 may determine whether the age of the target user is greater than or not less than a preset age (also referred to as a second preset age). If the age of the target user is greater than or not less than the second preset age, the processor 13 may determine that the physiological description data meets the second classification condition. However, if the age of the target user is not greater than or less than the second preset age, the processor 13 may determine that the physiological description data does not meet the second classification condition. For example, the second preset age may be 50 years old. It should be noted that the second preset age may also be adjusted according to practical requirements, and the disclosure is not limited thereto.

In one embodiment, the processor 13 may also confirm whether the physiological description data meets the first classification condition and/or the second classification condition according to the gender of the target user. In one embodiment, the processor 13 may also refer to the age and gender of the target user to confirm whether the physiological description data meets the first classification condition and/or the second classification condition. For example, the processor 13 may determine that the physiological description data meets the first classification condition and/or the second classification condition only when the age and gender of the target user meet the set conditions. In one embodiment, the processor 13 may also confirm whether the physiological description data meets the aforementioned first classification condition and/or the second classification condition according to the user identity identification information (or the physiological description data) in combination with other established decision rules.

In one embodiment, the detection result (i.e., the first detection result) obtained by evaluating the physiological structure status of the target user based on the first evaluation rule may include a detection value (also referred to as a first type detection value). For example, the first type detection value may be a T value (also referred to as a T-score). In one embodiment, the processor 13 may generate an online detection report corresponding to the image data according to the first type detection value.

In one embodiment, the processor 13 may compare the first type detection value with a threshold value (also referred to as a first threshold value) to obtain a comparison result (also referred to as a first comparison result). The first comparison result may reflect the relative relationship between the first type detection value and the first threshold value. For example, the first comparison result may reflect that the first type detection value is greater than, less than, or equal to the first threshold value.

In one embodiment, the processor 13 may generate an online detection report corresponding to the image data according to the first comparison result. For example, if the first comparison result reflects that the first type detection value is less than the first threshold value, the generated online detection report may reflect that the physiological structure status (e.g., bone density) of the target user may be abnormal. Alternatively, if the first comparison result reflects that the first type detection value is not less than the first threshold value, the generated online detection report may reflect that the physiological structure status (e.g., bone density) of the target user may be normal. In one embodiment, assuming that the physiological structure status of the target user is bone density, the first threshold value may be 2.5. It should be noted that the first threshold value may be adjusted according to practical requirements, and the disclosure is not limited thereto.

In one embodiment, the detection result (i.e., the second detection result) obtained by evaluating the physiological structure status of the target user based on the second evaluation rule may include another detection value (also referred to as a second type detection value). For example, the second type detection value may be a Z value (also referred to as a Z-score). In one embodiment, the processor 13 may generate an online detection report corresponding to the image data according to the second type detection value.

In one embodiment, the processor 13 may compare the second type detection value with a threshold value (also referred to as a second threshold value) to obtain a comparison result (also referred to as a second comparison result). The second comparison result may reflect the relative relationship between the second type detection value and the second threshold value. For example, the second comparison result may reflect that the second type detection value is greater than, less than, or equal to the second threshold value.

In one embodiment, the processor 13 may generate an online detection report corresponding to the image data according to the second comparison result. For example, if the second comparison result reflects that the second type detection value is less than the second threshold value, the generated online detection report may reflect that the physiological structure status (e.g., bone density) of the target user may be abnormal. Alternatively, if the second comparison result reflects that the second type detection value is not less than the second threshold value, the generated online detection report may reflect that the physiological structure status (e.g., bone density) of the target user may be normal. In one embodiment, assuming that the physiological structure status of the target user is bone density, the second threshold value may be 2.0. It should be noted that the second threshold value may also be adjusted according to practical requirements, and the disclosure is not limited thereto.

In one embodiment, after automatically generating the online detection report, the processor 13 may transmit the online detection report to the terminal device via the communication interface 11. The terminal device may present the online detection report through the display of the terminal device, so that a user of the terminal device may view the content of the online detection report.

In one embodiment, after detecting the upload request, in response to the upload request and before confirming whether the image data meets the automatic detection condition, the processor 13 may pre-execute an initialization operation (also referred to as an initialization procedure) corresponding to the automatic detection model. The initialization operation may be configured to set the automatic detection model 101 to a standby state. For example, the initialization operation may include pre-loading at least part of the program code and/or environment parameters required for the automatic detection model 101 to perform the automatic detection.

In one embodiment, in a standby state (i.e., a state in which at least part of the program code and/or environmental parameters required to perform the automatic detection have been pre-loaded), after confirming that the image data meets the automatic detection condition, the processor 13 may skip the initialization operation and directly perform automatic detection on the image data through the automatic detection model 101 in the standby state. Thereby, the operation efficiency of the automatic detection model 101 may be effectively improved, for example, the time spent by the automatic detection model 101 for performing automatic detection on the image data may be effectively reduced.

In one embodiment, after setting the automatic detection model 101 to the standby state, the processor 13 may continuously monitor the idle time of the automatic detection model 101. For example, during the idle time, the automatic detection model 101 is not enabled to perform the automatic detection on the image data. As the time that the automatic detection model 101 is in the standby state increases, the idle time may gradually increase. However, if the automatic detection model 101 is enabled at a certain time point to perform the automatic detection on the image data, the processor 13 may reset the idle time (e.g., resetting the idle time to zero).

In one embodiment, the processor 13 may determine whether the idle time meets a preset time length (also referred to as a first preset time length). If the idle time meets (e.g., is equal to) the first preset time length, it means that the automatic detection model 101 may have been idle for too long, resulting in at least part of the previously pre-loaded program code and/or environmental parameters being released. Therefore, the processor 13 may execute the initialization operation corresponding to the automatic detection model 101 again to restore the automatic detection model 101 to the standby state again. However, if the idle time does not meet (e.g., is not equal to) the first preset time length, the processor 13 may not execute the initialization operation corresponding to the automatic detection model 101 again. In one embodiment, whenever the idle time elapsed meets (e.g. is equal to) the first preset time length, the processor 13 may periodically execute the initialization operation corresponding to the automatic detection model 101 to maintain the automatic detection model 101 in the standby state.

In one embodiment, the processor 13 may further determine whether the idle time meets another preset time length (also referred to as a second preset time length). For example, the second preset time length may be longer than n times the first preset time length, and n is greater than 1. For example, if n is 10, it means that the second preset time length may be longer than 10 times the first preset time length. If the idle time meets (e.g. is equal to) the second preset time length, it means that the automatic detection model 101 has been idle for too long. Therefore, the processor 13 may stop executing the initialization operation corresponding to the automatic detection model 101 to release the hardware resources pre-occupied by the automatic detection model 101 for performing the automatic detection. However, if the idle time does not meet (e.g., is not equal to) the second preset time length, the processor 13 may continue to periodically execute the initialization operation corresponding to the automatic detection model 101. Thus, an optimal balance may be achieved between accelerating the startup of the automatic detection model 101 and reducing the hardware resource consumption.

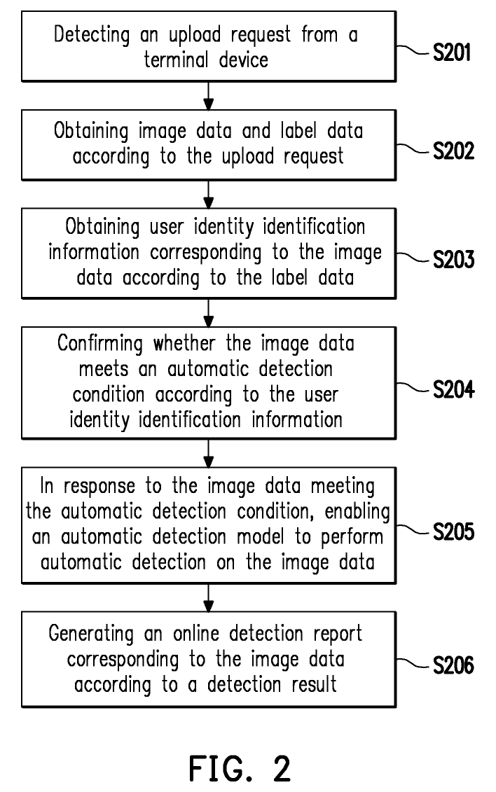

FIG. 2 is a flowchart of an online report generation method according to an embodiment of the disclosure. Referring to FIG. 2, in step S201, an upload request from a terminal device is detected, in which the upload request is configured to upload image data and label data. In step S202, the image data and the label data are obtained according to the upload request. In step S203, user identity identification information corresponding to the image data is obtained according to the label data. In step S204, whether the image data meets an automatic detection condition is confirmed according to the user identity identification information. In step S205, in response to the image data meeting the automatic detection condition, the automatic detection model is enabled to perform automatic detection on the image data. In step S206, an online detection report corresponding to the image data is generated according to a detection result.

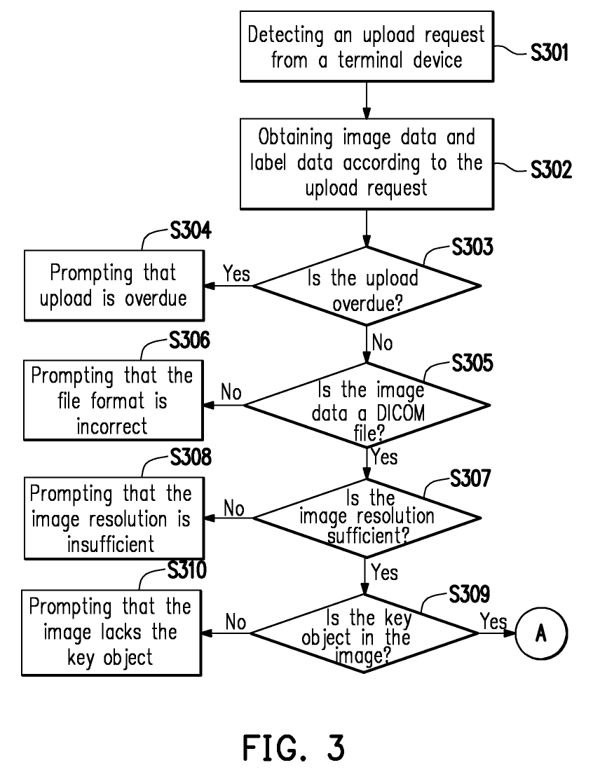

FIG. 3 is a flowchart of an online report generation method according to an embodiment of the disclosure. Referring to FIG. 3, in step S301, an upload request from a terminal device is detected, in which the upload request is configured to upload image data and label data. In step S302, the image data and the label data are obtained according to the upload request. In step S303, whether an upload overdue event corresponding to the image data occurs is determined. If the upload overdue event occurs, in step S304, a first prompt message is generated to remind that the upload overdue event corresponding to the image data has occurred, and the processing of the image data is suspended.

If the upload overdue event does not occur, in step S305, whether the file format of the image data complies with the specifications is determined, for example, whether the image data is a DICOM file is determined. If the file format of the image data does not comply with the specifications (e.g., the image data is not a DICOM file), in step S306, a second prompt message is generated to remind that the file format of the currently uploaded image data does not comply with the specifications, and the processing of the image data is suspended.

If the file format of the image data complies with the specifications (e.g., the image data is a DICOM file), in step S307, whether the image resolution of the image data is sufficient is determined. If the image resolution of the image data is insufficient, in step S308, a third prompt message is generated to remind that the image resolution of the currently uploaded image data is insufficient, and the processing of the image data is suspended.

If the image resolution of the image data is sufficient, in step S309, whether a key object is in the image data is determined. If the key object is not in the image data, in step S310, a fourth prompt message is generated to remind that the required key object is not in the currently uploaded image data, and the processing of the image data is suspended.

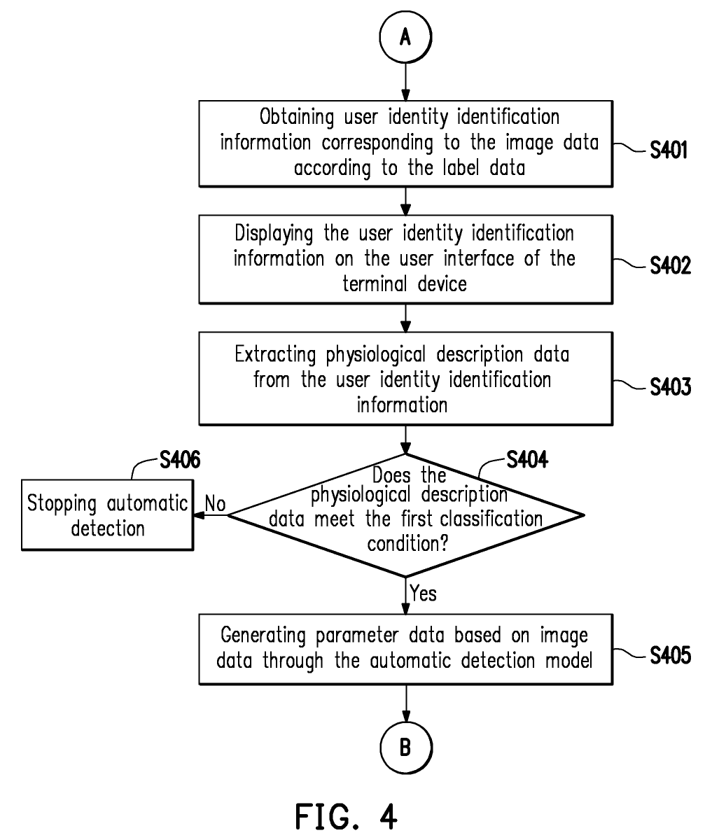

If the key object is in the image data, the process proceeds to step S401 in FIG. 4. It should be noted that, in the embodiment of FIG. 3, the execution order of steps S303, S305, S307 and S309 may be adjusted according to practical requirements, and the disclosure is not limited thereto.

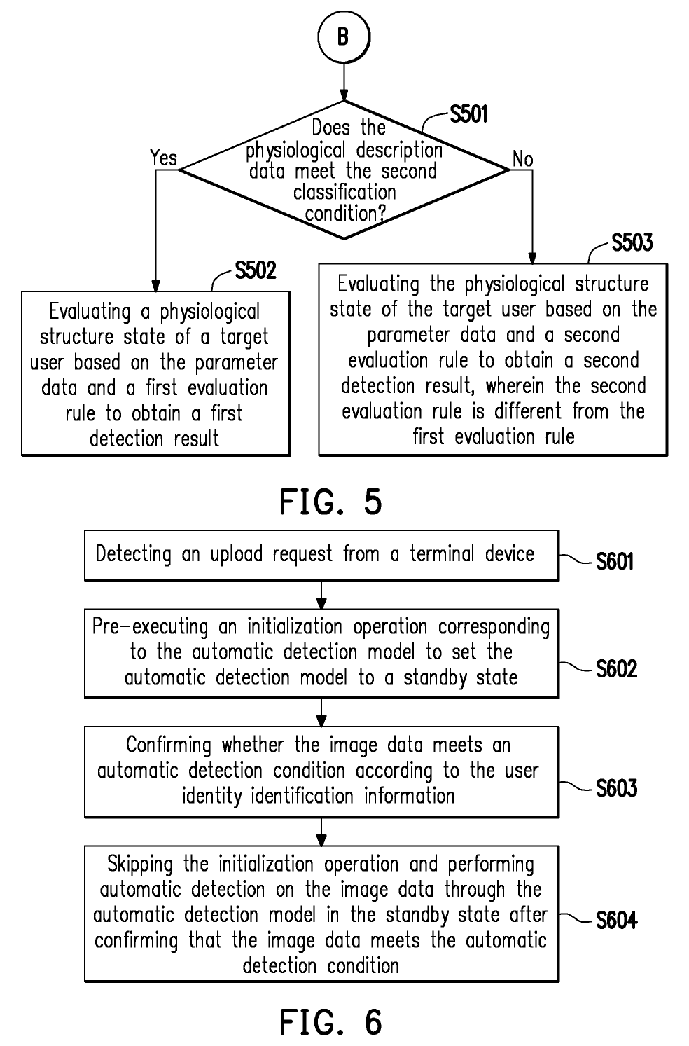

FIG. 4 is a flowchart of an online report generation method according to an embodiment of the disclosure. Referring to FIG. 4, in step S401, user identity identification information corresponding to the image data is obtained according to the label data. In step S402, the user identity identification information is displayed on the operation interface of the terminal device. In step S403, physiological description data is extracted from the user identity identification information. In step S404, whether the physiological description data meets a first classification condition is determined. If the physiological description data meets the first classification condition, in step S405, the automatic detection model generates parameter data based on the image data. Then, the process may proceed to step S501 in FIG. 5. However, if the physiological description data does not meet the first classification condition, in step S406, the automatic detection of the image data is stopped.

FIG. 5 is a flowchart of an online report generation method according to an embodiment of the disclosure. Referring to FIG. 5, in step S501, whether the physiological description data meets a second classification condition is determined. If the physiological description data meets the second classification condition, in step S502, a physiological structure status of the target user is evaluated based on the parameter data and a first evaluation rule among multiple evaluation rules to obtain a first detection result. However, if the physiological description data does not meet the second classification condition, in step S503, the physiological structure status of the target user is evaluated based on the parameter data and a second evaluation rule among the multiple evaluation rules to obtain a second detection result. The second evaluation rule is different from the first evaluation rule.

FIG. 6 is a flowchart of an online report generation method according to an embodiment of the disclosure. Referring to FIG. 6, in step S601, an upload request from a terminal device is detected. Before confirming whether the image data meets the automatic detection condition, in step S602, an initialization operation corresponding to the automatic detection model is pre-executed to set the automatic detection model to a standby state. In step S603, whether the image data meets an automatic detection condition is confirmed according to the user identity identification information. In step S604, after confirming that the image data meets the automatic detection condition, the initialization operation is skipped and the automatic detection model in the standby state performs automatic detection on the image data.

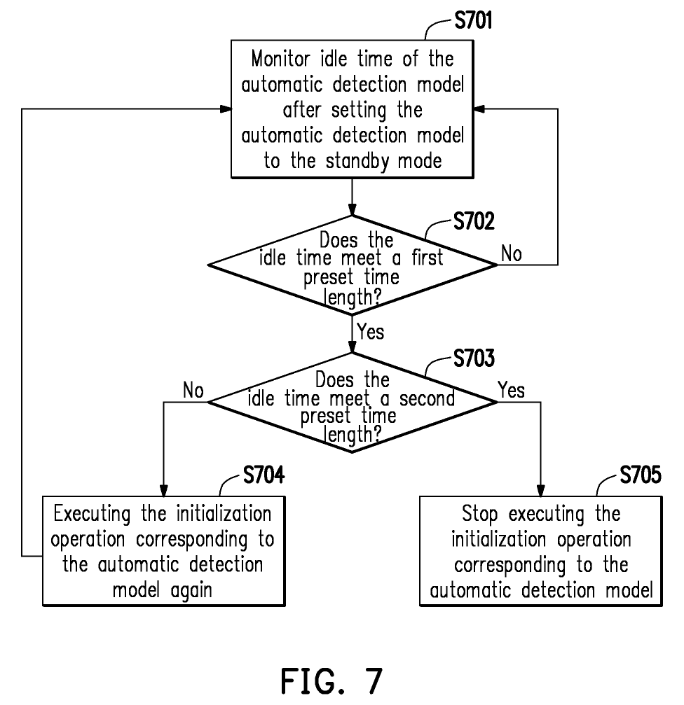

FIG. 7 is a flowchart of an online report generation method according to an embodiment of the disclosure. Referring to FIG. 7, in step S701, after setting the automatic detection model to the standby state, the idle time of the automatic detection model is continuously monitored. In step S702, whether the idle time meets a first preset time length is determined. If the idle time does not meet the first preset time length, return to step S701. If the idle time meets the first preset time length, in step S703, whether the idle time meets the second preset time length is determined.

If the idle time meets the first preset time length but does not meet the second preset time length, in step S704, the initialization operation corresponding to the automatic detection model is executed again to restore or maintain the automatic detection model in the standby state. However, if the idle time meets the second preset time length, in step S705, the execution of the initialization operation corresponding to the automatic detection model is stopped.

However, each step in FIG. 2 to FIG. 7 has been described in detail as the above, and are not repeated herein. It should be noted that each step in FIG. 2 to FIG. 7 may be implemented as multiple codes or circuits, which is not limited by the disclosure. In addition, the methods of FIG. 2 to FIG. 7 may be used in conjunction with the above exemplary embodiments, or may also be used alone, which is not limited by the disclosure.

In summary, the online report generation method and the electronic device provided in the disclosure include a comprehensive and efficient online report generation solution to assist professionals or non-professionals in evaluating the physiological structure status of the user. In addition, the online report generation method and the electronic device provided in the disclosure may also perform real-time and continuous performance optimization for the automatic detection model, thereby improving the overall operational efficiency of the system.

Although the disclosure has been described in detail with reference to the above embodiments, they are not intended to limit the disclosure. Those skilled in the art should understand that it is possible to make changes and modifications without departing from the spirit and scope of the disclosure. Therefore, the protection scope of the disclosure shall be defined by the following claims.

What is claimed is:

1. An online report generation method, comprising:
detecting an upload request from a terminal device, wherein the upload request is configured to upload image data and label data;
obtaining the image data and the label data according to the upload request;
obtaining user identity identification information corresponding to the image data according to the label data;
confirming whether the image data meets an automatic detection condition according to the user identity identification information;
in response to the image data meeting the automatic detection condition, enabling an automatic detection model to perform automatic detection on the image data; and
generating an online detection report corresponding to the image data according to a detection result.

2. The online report generation method according to claim 1, wherein the image data and the label data comply with a digital imaging and communications in medicine (DICOM) protocol.

3. The online report generation method according to claim 1, wherein obtaining the user identity identification information corresponding to the image data according to the label data comprises:
confirming a data parsing format corresponding to the label data according to the upload request; and
extracting the user identity identification information from the label data based on the data parsing format.

4. The online report generation method according to claim 1, wherein the user identity identification information comprises physiological description data related to a target user.

5. The online report generation method according to claim 1, wherein confirming whether the image data meets the automatic detection condition according to the user identity identification information comprises:
confirming whether the physiological description data related to a target user meets a first classification condition according to the user identity identification information;
determining that the image data meets the automatic detection condition if the physiological description data meets the first classification condition; and
determining that the image data does not meet the automatic detection condition if the physiological description data does not meet the first classification condition.

6. The online report generation method according to claim 1, wherein the automatic detection comprises:
generating parameter data through the automatic detection model based on the image data, wherein the parameter data reflects a physiological structure status of a target user; and
inputting the parameter data into a hierarchical evaluation model according to the user identity identification information to obtain the detection result, wherein the detection result reflects an evaluation result of the physiological structure status based on at least one of a plurality of evaluation rules.

7. The online report generation method according to claim 6, wherein inputting the parameter data into the hierarchical evaluation model according to the user identity identification information to obtain the detection result comprises:
confirming whether physiological description data of the target user meets a second classification condition according to the user identity identification information;
evaluating the physiological structure status based on the parameter data and a first evaluation rule among the plurality of evaluation rules to obtain a first detection result if the physiological description data of the target user meets the second classification condition; and
evaluating the physiological structure status based on the parameter data and a second evaluation rule among the plurality of evaluation rules to obtain a second detection result if the physiological description data of the target user does not meet the second classification condition, wherein the second evaluation rule is different from the first evaluation rule.

8. The online report generation method according to claim 1, further comprising:
in response to the upload request, pre-executing an initialization operation corresponding to the automatic detection model to set the automatic detection model to a standby state before confirming whether the image data meets the automatic detection condition,
wherein in response to the image data meeting the automatic detection condition, enabling the automatic detection model to perform the automatic detection on the image data comprises:
skipping the initialization operation, and performing the automatic detection on the image data through the automatic detection model in the standby state after confirming that the image data meets the automatic detection condition.

9. The online report generation method according to claim 8, further comprising:
monitoring idle time of the automatic detection model after setting the automatic detection model to the standby state; and
executing the initialization operation corresponding to the automatic detection model again if the idle time meets a first preset time length.

10. The online report generation method according to claim 9, further comprising:
stop executing the initialization operation corresponding to the automatic detection model if the idle time meets a second preset time length, wherein the second preset time length is longer than n times the first preset time length, and n is greater than 1.

11. An electronic device, comprising:
a storage device, configured to store an automatic detection model; and
a processor, coupled to the storage device,
wherein the processor is configured to:
detect an upload request from a terminal device, wherein the upload request is configured to upload image data and label data;
obtain the image data and the label data according to the upload request;
obtain user identity identification information corresponding to the image data according to the label data;
confirm whether the image data meets an automatic detection condition according to the user identity identification information;
in response to the image data meeting the automatic detection condition, enable the automatic detection model to perform automatic detection on the image data; and
generate an online detection report corresponding to the image data according to a detection result.

12. The electronic device according to claim 11, wherein the image data and the label data comply with a digital imaging and communications in medicine (DICOM) protocol.

13. The electronic device according to claim 11, wherein the processor obtaining the user identity identification information corresponding to the image data according to the label data comprises:
confirming a data parsing format corresponding to the label data according to the upload request; and
extracting the user identity identification information from the label data based on the data parsing format.

14. The electronic device according to claim 11, wherein the user identity identification information comprises physiological description data related to a target user.

15. The electronic device according to claim 11, wherein the processor confirming whether the image data meets the automatic detection condition according to the user identity identification information comprises:
confirming whether the physiological description data related to a target user meets a first classification condition according to the user identity identification information;
determining that the image data meets the automatic detection condition if the physiological description data meets the first classification condition; and
determining that the image data does not meet the automatic detection condition if the physiological description data does not meet the first classification condition.

16. The electronic device according to claim 11, wherein the automatic detection comprises:
generating parameter data through the automatic detection model based on the image data, wherein the parameter data reflects a physiological structure status of a target user; and
inputting the parameter data into a hierarchical evaluation model according to the user identity identification information to obtain the detection result, wherein the detection result reflects an evaluation result of the physiological structure status based on at least one of a plurality of evaluation rules.

17. The electronic device according to claim 16, wherein the processor inputting the parameter data into the hierarchical evaluation model to obtain the detection result according to the user identity identification information comprises:
confirming whether physiological description data of the target user meets a second classification condition according to the user identity identification information;
evaluating the physiological structure status based on the parameter data and a first evaluation rule among the plurality of evaluation rules to obtain a first detection result if the physiological description data of the target user meets the second classification condition; and
evaluating the physiological structure status based on the parameter data and a second evaluation rule among the plurality of evaluation rules to obtain a second detection result if the physiological description data of the target user does not meet the second classification condition, wherein the second evaluation rule is different from the first evaluation rule.

18. The electronic device according to claim 11, wherein the processor further comprises:
in response to the upload request, pre-executing an initialization operation corresponding to the automatic detection model to set the automatic detection model to a standby state before confirming whether the image data meets the automatic detection condition,
wherein in response to the image data meeting the automatic detection condition, the processor enabling the automatic detection model to perform the automatic detection on the image data comprises:
skipping the initialization operation, and performing the automatic detection on the image data through the automatic detection model in the standby state after confirming that the image data meets the automatic detection condition.

19. The electronic device according to claim 18, wherein the processor further comprises:
monitoring idle time of the automatic detection model after setting the automatic detection model to the standby state; and
executing the initialization operation corresponding to the automatic detection model again if the idle time meets a first preset time length.

20. The electronic device according to claim 19, wherein the processor further comprises:
stop executing the initialization operation corresponding to the automatic detection model if the idle time meets a second preset time length, wherein the second preset time length is longer than n times the first preset time length, and n is greater than 1.
